(19) United States
(12) Patent Application Publication
(10) Pub. No.: US 2025/0011849 A1
(43) Pub. Date: Jan. 9, 2025

(54) DIGITAL DROPLET PCR SYSTEM

(71) Applicant: Hewlett-Packard Development Company, L.P., Spring, TX (US)

(72) Inventors: Michael Cumbie, Corvallis, OR (US); Viktor Shkolnikov, Palo Alto, CA (US)

(73) Assignee: Hewlett-Packard Development Company, L.P., Spring, TX (US)

(21) Appl. No.: 18/702,182

(22) PCT Filed: Oct. 22, 2021
(86) PCT No.: PCT/US2021/056135

§ 371 Date: Apr. 17, 2024

(51) Int. Cl. C12Q 1/6844; B01L 3/00; B01L 7/00

(52) U.S. Cl. CPC C12Q 1/6844; B01L 3/50273; B01L 7/52; B01L 2200/0673; B01L 2200/16; B01L 2300/0654; B01L 2300/18

(57) ABSTRACT

A digital droplet PCR system is described. The digital droplet PCR system comprises a microfluidic cartridge comprising: a plurality of sample droplet generators, wherein each sample droplet generator is operable to partition a PCR mixture into a plurality of aqueous droplets dispersed in a carrier liquid, and wherein at least one sample droplet generator is operable to partition a PCR mixture that is different to a PCR mixture that is partitioned by at least one other sample droplet generator; a thermocycling chamber comprising an embedded heater, an inlet configured to receive the plurality of aqueous droplets from the plurality of sample droplet generators, and an outlet; and an optical readout zone fluidly connected to the outlet of the thermocycling chamber; and a pressure actuated pump configured to couple to and cause fluid flow through the microfluidic cartridge. A method of performing digital droplet PCR is also described.

### BACKGROUND

The Polymerase Chain Reaction (PCR) is used to amplify specific nucleic acid sequences and detect their presence in a sample. PCR can be used for many different applications, including quantification of gene expression, patient genotyping and also as a diagnostic tool to identify the presence of one or more pathogens, for example bacteria or viruses in a sample from a patient by amplifying and detecting nucleic acid sequences that are specific to a particular pathogen. Personalised medicine requires genotyping using PCR in which the detection of one or more biomarkers, for example specific mutations, may influence clinical decisions on the nature or type of medical intervention.

PCR subjects a sample to amplification conditions in the presence of an enzyme capable of elongating nucleic acid strands, for example a polymerase. The three basic steps of a single round or cycle of PCR amplification are denaturation, annealing and chain extension, each optimally taking place at different temperatures (for example in the ranges of 94-98° C. for denaturation; 50-65° C. for annealing, and 70-80° C. for chain extension, depending on polymerase), with each set of three steps being known by the term “thermocycling”. The amplification products (amplicons) are detected optically, for example using fluorescent reporters.

Digital droplet PCR (ddPCR) is based on forming a water-in-oil emulsion, with each droplet having as little as one copy of the nucleic acid sample (based on dilution and statistical probability), along with the PCR reagents. Each droplet serves as a self-contained micro-reactor for a PCR amplification.

### BRIEF DESCRIPTION OF THE DRAWINGS

FIG. 1 is a schematic of an example ddPCR system;

FIG. 2 shows an example thermocycling chamber of a microfluidic cartridge;

FIG. 3A shows in plan view an example flow channel of a microfluidic cartridge;

FIG. 3B shows a cross-section view the example flow channel of FIG. 3A;

FIG. 4 shows in plan view an example flow channel of a microfluidic cartridge;

FIG. 5 shows in plan view an example flow channel of a microfluidic cartridge;

FIG. 6 shows in plan view an example flow channel of a microfluidic cartridge;

FIG. 7 shows in plan view an example flow channel of a microfluidic cartridge;

FIG. 8A shows in plan view an example flow channel of a microfluidic cartridge; and

FIG. 8B shows a cross-section view the example flow channel of FIG. 8A.

### DETAILED DESCRIPTION

Before particular embodiments of the present method and other aspects are disclosed and described, it is to be understood that the present method and other aspects are not limited to the particular process and materials disclosed herein as such may vary to some degree. It is also to be understood that the terminology used herein is used for the purpose of describing particular embodiments only and is not intended to be limiting, as the scope of the present method and other aspects will be defined only by the appended claims and equivalents thereof.

In the present specification, and in the appended claims, the following terminology will be used:

The singular forms “a”, “an” and “the” include plural referents unless the context clearly dictates otherwise. Thus, for example, reference to “a sensor” includes reference to one or more of such sensors.

The terms “about” and “approximately” when referring to a numerical value or range is intended to encompass the values resulting from experimental error that can occur when taking and/or making measurements.

Concentrations, amounts, and other numerical data may be presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of approximately 1 wt. % to approximately 20 wt. % should be interpreted to include not only the explicitly recited concentration limits of 1 wt. % to approximately 20 wt. %, but also to include individual concentrations such as 2 wt. %, 3 wt. %, 4 wt. %, and sub-ranges such as 5 wt. % to 10 wt. %, 10 wt. % to 20 wt. %, etc.

In the following description, for purposes of explanation, numerous specific details are set forth in order to provide a thorough understanding of the present apparatus and methods. It will be apparent, however, to one skilled in the art, that the present apparatus and methods maybe practiced without these specific details. Reference in the specification to “one example” or “an example” means that a particular feature, structure, or characteristic described in connection with the example is included in at least one example. The appearance of the phrase “in one example” in various places in the specification are not necessarily all referring to the same example.

Unless otherwise stated, any feature described herein can be combined with any aspect or any other feature described herein.

As used herein, the abbreviations “PCR”, “dNTPs” and “primers” refer to the “Polymerase Chain Reaction” and its components. Specifically, the term “dNTP” refers to the 2′-deoxynucleotide triphosphates used in PCR. The four standard dNTPs are 2′-deoxyadenosine 5′-triphosphate, 2′-deoxyguanosine 5′-triphosphate, 2′-deoxycytosine 5′-triphosphate and thymidine 5′-triphosphate (already lacking a 2′-hydroxyl), though modified dNTPs, for example non-natural nucleotides incorporating labels or reporter molecules, or reactive moieties may also be used (for example in the form of nucleobase modifications such as C7-modified deaza-guanine or C7-modified deaza-adenine or C5-modified cytosine or C5-modified thymidine). Non-natural nucleotides having for example sugar modifications (such as 2′-F or 2′-OMe modifications, or “LNA”, having an O—CH₂ linker between the 2′ and 4′ positions on the sugar), or backbone modifications such as phosphorothioates, or phosphorothiolates may also be used, as may unnatural bases such as the pyrimidine analog 6-amino-5-nitro-3-(1′-β-D-2′-deoxyribofuranosyl)-2(1H)-pyridone (dZ) and its Watson-Crick complement, the purine analog 2-amino-8-(1′-β-D-2′-deoxyribofuranosyl)-imidazo[1,2-a]-1,3,5-triazin-4(8H)-one (dP).

As used herein, the term “primer” refers to a short single stranded nucleic acid, for example an oligodeoxynucleotide (also referred to as an oligonucleotide herein), of about 15 to 30 nucleotides in length. A primer is designed to base pair in a specific or complementary manner to a nucleic acid sequence of interest, and so is considered specific to that nucleic acid. DNA is directional, with the 3′ end of one strand forming base pairs with the 5′-end of the counter strand and a primer is usually designed so that its 5′-end base pairs to the 3′-end of the nucleic acid of interest so that DNA synthesis (which occurs in a 5′ to 3′ direction) to elongate the primer can occur.

As used herein, the terms “oligonucleotide pair”, “oligonucleotide primer pair” and “primer pair” refer to a set of two oligonucleotides that can serve as forward and reverse primers for a nucleic acid of interest. As both strands are copied and amplified in a PCR reaction, each strand requires a primer: the forward primer attaches to the start codon of the template DNA strand (the anti-sense strand), while the reverse primer attaches to the stop codon of the complementary strand of DNA (the sense strand). The 5′-end of each primer binds to the 3′-end of the complementary DNA strand of the nucleic acid of interest.

As used herein, the term “nucleic acid of interest”, or “target”, refers to a polynucleotide sequence, for example of at least one hundred, two hundred, three hundred, four hundred, five hundred or up to one thousand nucleotides in length. The polynucleotide sequence may be specific to a particular organism such as a pathogen, or may be suspected of having a particular mutation along its length, and will encode a particular polypeptide or protein, or mutant form thereof. For example, the polynucleotide sequence may encode the spike protein of SARS-CoV-2, or may encode a mutant form of the epidermal growth factor receptor (EGFR) the presence or absence of which renders a patient more or less likely to respond well to cancer treatments such as erlotinib or gefitinib.

Regardless of end application, PCR subjects a sample to multiple rounds of thermocycling in the presence of an enzyme capable of elongating nucleic acid strands, for example a polymerase. Polymerases catalyse the reaction between a deoxynucleotide triphosphate and a DNA strand, producing an elongated DNA strand bearing one more nucleotide (from the deoxynucleotide triphosphate), and pyrophosphate as a by-product. Examples of polymerases used in PCR are thermostable polymerases such as Taq polymerase (from Thermus aquaticus), Pfu polymerase (from Pyrococcus furiosus), and Bst polymerase (from Bacillus stearothermophilus). The DNA strand that is elongated in PCR is usually in the form of an oligonucleotide primer specific to a target nucleic acid sequence of interest, which is elongated using a mixture of deoxyribonucleotide triphosphates (dNTPs). For full synthesis of a standard DNA strand, four dNTPs corresponding to the four nucleobases found in DNA (adenine, guanosine, thymine and cytosine) are required: 2′-deoxyadenosine 5′-triphosphate, 2′-deoxyguanosine 5′-triphosphate, 2′-deoxycytosine 5′-triphosphate and thymidine 5′-triphosphate.

The three basic steps of a single round or cycle of PCR amplification are denaturation, annealing and chain extension, each optimally taking place at different temperatures (for example in the ranges 94-98° C. for denaturation; 50-65° C. for annealing, and 70-80° C. for chain extension, depending on polymerase), hence the term thermocycling. The denaturation step separates the two strands of double-stranded DNA, with each strand acting as a template in the later chain extension step in which a complete complementary strand to the template is produced.

An oligonucleotide primer (comprising for example 15 to 30 nucleotides to ensure a balance of good specificity and efficient hybridization) is annealed to the 3′-end of each single stranded DNA molecule, and acts as a template for the synthesis of the new strand. A DNA polymerase, and a mix of dNTPs then synthesize the new strand in the chain extension step, using the original single strand of DNA as its template. Since both strands of the original DNA duplex are used as templates, a singe round or cycle of PCR results in a doubling of the number of DNA duplexes. The number of copies thus increases exponentially with the number of cycles of amplification: after 2 cycles, four DNA duplexes are present in the sample, while after 3 cycles, 8 duplexes are present.

PCR can be performed on a prepared sample of 10-50 μL and quantified by monitoring the fluorescence of the fluid as it is thermally cycled. Since the fluorescence is proportional to the amount of nucleic acid (double stranded DNA), the fluorescence intensity increases as the number of cycles of amplification (the amount of double stranded DNA produced) increases. However, in order to achieve a high enough signal to noise ratio, 40 cycles or more are required.

The amplification products (amplicons) are detected optically, for example using fluorescent reporters. Fluorescent reporter molecules used in PCR include non-specific fluorescent dyes, such as SYBR Green I, which has a distinct emission spectrum when intercalated into any double-stranded DNA, leading to an increase in fluorescence as more double-stranded DNA is produced. Other suitable reporter molecules include target-specific fluorescent reporter molecules, such as the TaqMan hydrolysis probes of target-specific nucleic acids labelled with fluorescent reporter and quencher, with the probe being hydrolyzed by the exonuclease activity of the Taq polymerase, releasing the reporter from the quencher and again leading to an increase in fluorescence.

Reporter molecules may also be linked to a primer to be used in the PCR amplification, such as in the Scorpion® system, a single-stranded bi-labeled fluorescent probe sequence forming a hairpin-loop conformation with a 5′ end reporter and an internal quencher directly linked to the 5′ end of a primer via a blocker (which prevents the polymerase from extending the primer). In the beginning, the polymerase extends the primer and synthesizes the complementary strand of the specific target sequence. During the next cycle, the hairpin-loop unfolds and the loop-region of the probe hybridizes intramolecularly to the newly synthesized target sequence. Now that the reporter is no longer in close proximity to the quencher, fluorescence emission may take place. The fluorescent signal is detected and is directly proportional to the amount of amplified nucleic acid.

Since different reporter dyes have different, and distinct emission spectra, combinations of different reporters can be strategically used in multiplex reactions. In addition to SYBR Green I, other cyanine dyes such as Cy3, or Cy5 can be used, as well as rhodamine dyes. Cy3 has a fluorescence emission at 570 nm, while Cy5 has a fluorescence emission at 670 nm. Other reporter dyes include the Alexa Fluor series of dyes, which have emission wavelengths ranging from 440 nm to 805 nm.

Multiplex PCR is a technique used for amplification of multiple, different, nucleic acid sequences of interest in a single experiment. For example, multiplex PCR may be used to screen for the presence of nucleic acid sequences of interest from multiple, different pathogens in a single reaction, such as simultaneously screening a single sample for the presence of viral nucleic acid sequences from any of SARS-CoV, MERS, SARS-CoV-2, influenza, and Ebola viruses.

In a multiplex PCR, many different primer pairs are required, with each pair specific to a nucleic acid sequence of interest. For example, if a sample of nucleic acid was being investigated for the presence of 10 different specific nucleic acid sequences of interest (for example 10 different viruses, or 10 different genetic mutations in a patient), then at least 10 different primer pairs would be required for the multiplex PCR.

There is a drive toward the development of new PCR systems, which could be used at the point of care/field of use. Such systems would need rapid thermocycling, in order to amplify the nucleic acid sequences of interest and quickly provide a positive/negative test result.

The present inventors have sought to address these challenges by providing a PCR system capable of multiplexing with rapid thermocycling capabilities.

In one example there is provided a digital droplet PCR system, comprising:
a microfluidic cartridge comprising:
   a plurality of sample droplet generators, wherein each sample droplet generator is operable to partition a PCR mixture into a plurality of aqueous droplets dispersed in a carrier liquid, and wherein at least one sample droplet generator is operable to partition a PCR mixture that is different to a PCR mixture that is partitioned by at least one other sample droplet generator;
   a thermocycling chamber comprising an embedded heater, an inlet configured to receive the plurality of aqueous droplets from the plurality of sample droplet generators, and an outlet; and
   an optical readout zone fluidly connected to the outlet of the thermocycling chamber; and
a pressure-actuated pump configured to couple to and cause fluid flow through the microfluidic cartridge.

In a further example there is provided a method of performing PCR, comprising:
partitioning, in a microfluidic cartridge, a first PCR mixture into a plurality of aqueous droplets and dispersing the plurality of aqueous droplets in a carrier liquid to form a first PCR dispersion;
transporting the first PCR dispersion into a thermocycling chamber of the microfluidic cartridge;
partitioning, in the microfluidic cartridge, a second PCR mixture into a plurality of aqueous droplets and dispersing the plurality of aqueous droplets in a carrier liquid to form a second PCR dispersion;
transporting the second PCR dispersion into the thermocycling chamber to form a reaction emulsion of first and second PCR dispersions;
subjecting the reaction emulsion to conditions suitable for amplification using polymerase chain reaction; and
successively transporting each droplet of the reaction emulsion from the thermocycling chamber to an optical readout zone of the microfluidic cartridge and detecting an optical signal from each droplet.

The system of the present disclosure enables a rapid method of multiplexed digital droplet PCR having rapid heating rates (˜160° C./s) and rapid cooling rates (30° C./s), allowing for rapid thermocycling and a short time to result (less than 20 minutes). The system also enables sensitive assays to be performed (less than 100 copies/mL of target detected), also exhibiting high specificity.

### PCR System

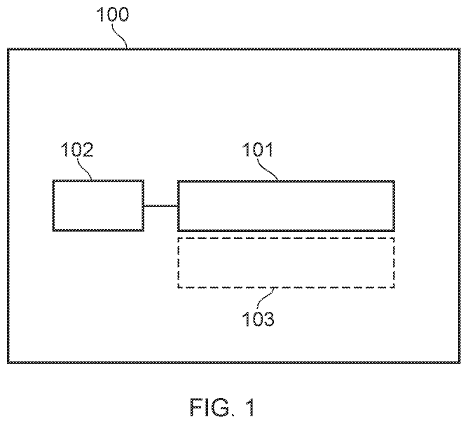

FIG. 1 schematically shows a PCR system 100 in accordance with the present disclosure. PCR system 100 comprises a microfluidic cartridge 101, a pressure-actuated pump 102 and an optional cooling module 103. Although not shown, PCR system 100 may further include electronic circuitry and processing capabilities. PCR amplification occurs in a thermocycling chamber provided in microfluidic cartridge 101 as will be described below.

PCR system 100 comprises a microfluidic cartridge comprising: a plurality of sample droplet generators, wherein each sample droplet generator is operable to partition a PCR mixture into a plurality of aqueous droplets dispersed in a carrier liquid, and wherein at least one sample droplet generator is operable to partition a PCR mixture that is different to a PCR mixture that is partitioned by at least one other sample droplet generator; a thermocycling chamber comprising an embedded heater, an inlet configured to receive the plurality of aqueous droplets from the plurality of sample droplet generators, and an outlet; and an optical readout zone fluidly connected to the outlet of the thermocycling chamber.

The microfluidic cartridge may comprise a single thermocycling chamber or a plurality of thermocycling chambers provided on a substrate. A plurality of thermocycling chambers may be independently operable. In other words, each thermocycling chamber of a plurality of thermocycling chambers may have its own dedicated heater or heaters, embedded in the cartridge, so as to be independently controllable. In this way, synchronous or asynchronous control of a plurality of different droplet PCR assays can be performed. Asynchronous control of individual thermocycling chambers within a plurality of thermocycling chambers enables the amplification of a plurality of different samples using different thermocycling, or even isothermal, protocols. In some examples, each thermocycling chamber is provided with a plurality of independently operable heaters or heating elements, thus enabling a plurality of different droplet PCR assays to be performed via different protocols in a single thermocycling chamber, as described in the methods of the present disclosure.

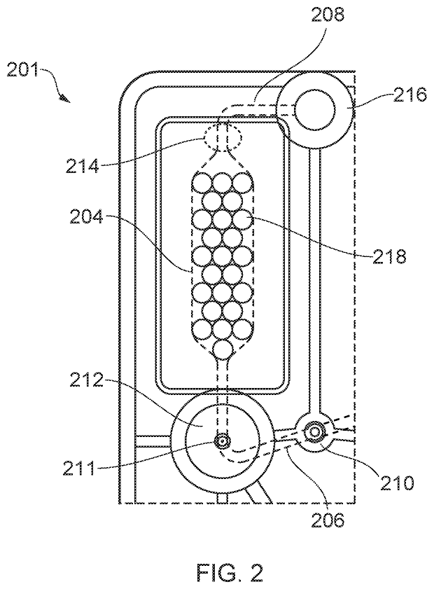

FIG. 2 shows a plan view of a region of a microfluidic cartridge 201 in accordance with the disclosure. While FIG. 2 shows one example of a microfluidic cartridge, other configurations are possible. For example, microfluidic cartridge 201 comprises a thermocycling chamber 204 fed by a flow channel 206. Before reaching thermocycling chamber 204, flow channel 206 interfaces with a sample droplet generator 210 fluidly connected to a sample reservoir (not shown). Thermocycling chamber 204 has an inlet configured to receive a plurality of aqueous droplets from sample droplet generator 210 by being coupled to flow channel 206, and an outlet opposite the inlet. Thermocycling chamber 204 is presented as an elongate chamber, with tapering sections at each end. At the end of thermocycling chamber 204 furthest from flow channel 206, an opening is provided to a flow channel 208 that leads to a waste reservoir 216. Flow channel 206, thermocycling chamber 204 and flow channel 208 are outlined with dashed lines, to reflect that these are present in a lower layer of microfluidic cartridge 201 than the other components that are outlined with solid lines.

A region of flow channel 208 that is indicated at reference 214 functions as an optical readout zone, on which an optical detector such as a fluorimeter will focus. Within thermocycling chamber 204, a plurality of droplets 218 are shown. For convenience, FIG. 2 shows a single sample droplet generator 210 being coupled to thermocycling chamber 204, although as will be shown in subsequent Figures, a plurality of sample droplet generators are present on microfluidic cartridge 201, connected to flow channel 206 upstream of sample droplet generator 210, out of the image.

Thermocycling chamber 204 and associated components shown in FIG. 2 may form part of a microfluidic layer disposed on a substrate of microfluidic cartridge 201. The substrate may be formed from any material suitable for microfluidics, such as glass, silicon, SU-8 (an epoxy-based photoresist material), or polycarbonate. A heater is embedded on or within the substrate, to provide heat to the thermocycling chamber. In some examples, the substrate comprises or is a printed circuit board (PCB), and so in some examples is termed a PCB substrate. In some examples, the heater comprises one or more printed electrical traces embedded on or in a substrate to provide heat to the reaction chamber, or one or more electrical traces etched from or into a conductive material. In some examples, the heater is provided above or below the plane of the microfluidic cartridge. In some examples, the heater is embedded into a substrate on which the thermocycling chamber is disposed. The heater may be embedded into the substrate by machining or etching portions of the substrate into which the heater can be embedded, or by encapsulating the heater in a liquid precursor material that can be solidified or cured to form the substrate. A suitable material is SU-8, which is a liquid, until cured by UV light. In other examples, the heater is provided on a surface of the substrate. In some examples, the heater comprises a flat panel heater or one or more thermally conductive printed or etched electrical traces. In some examples, the heater comprises a Peltier device, a flat panel heater in the form of a solid-state active heat pump. The heater may be formed of any thermally conductive material, such as copper, or gold or silver.

The heater may be in the form of a plurality of thermally conductive printed or etched electrical traces. The plurality of thermally conductive traces may be arranged parallel to each other in a single plane, and be spaced from each other in a horizontal direction by at least 5 μm, for example at least 10 μm, for example at least 50 μm, for example at least 100 μm, for example at least 200 μm. The plurality of thermally conductive traces may be spaced from each other by a distance in the range of from 5 μm to 200 μm, for example 10 μm to 100 μm.

In other examples, the plurality of thermally conductive traces may be arranged in different or multiple planes, and be termed a bi-layer or multi-layer heater. The traces of a bi-layer or multi-layer heater may be spaced apart in the vertical direction by at least 5 μm, for example at least 10 μm, for example at least 50 μm, for example at least 100 μm, for example at least 200 μm. Each layer may be spaced from another layer in the vertical direction by a distance in the range of from 5 μm to 200 μm, for example 10 μm to 100 μm.

In some examples the heater comprises at least partially overlapping conductive traces provided in different layers of the microfluidic cartridge. For example, the bi-layer or multi-layer heater may comprise a plurality of thermally conductive traces in which the traces of one layer run parallel to and at least partially or completely overlie the traces of a second layer.

Alternatively, the bi-layer or multi-layer heater may comprise a plurality of thermally conductive traces in which the traces of one layer run perpendicular or substantially perpendicular to the traces of a second layer. In other examples, the bi-layer or multi-layer heater may comprise a plurality of thermally conductive traces in which the traces of one layer run parallel to the traces of a second layer and partially overlap or do not overlap with the traces of the underlying layer. In this example, viewed from above or below the two planes, the two sets of traces may resemble interdigitated electrical traces. The heater may comprise a bi-layer or multi-layer configuration of electrical traces in which a series of printed or electrical traces are overlaid with a diffuser layer or heat spreader of thermally conductive material which acts to diffuse or spread heat from the traces to a fluid in the overlying thermocycling chamber. Thus, the heater can comprise a series of electrical traces in a first layer of the microfluidic cartridge overlaid with a diffuser of thermally conductive material in a second layer of the microfluidic cartridge.

The diffuser layer may be a passive heat spreader, and be formed from the same thermally conductive material as the thermally conductive material of the printed traces, for example deposited copper which can be etched into any required pattern. In some examples, one or more heater elements comprises a resistive heater. Etched copper traces are examples of resistive heaters, as they not only dissipate heat, but their resistance changes with temperature.

Resistive heaters of this type are particularly suited for use in the thermocycling chamber of the present disclosure as they can be used not only as heaters, but simultaneously also be used as temperature sensors to monitor temperature within the thermocycling chamber and allow immediate and accurate feedback control. Since copper does not affect a PCR reaction, it is also suited to be a heater in direct contact with a fluid within the thermocycling chamber, thereby further improving thermal transfer and heating rates. The heater may be in the form of one or more serpentine traces. It will be understood that the term “serpentine” refers to a single trace having a plurality of parallel sections joined at their ends to neighbouring sections. The heater may comprise more than one serpentine trace, in different planes of the substrate. In one configuration, an outer or upper serpentine trace may be provided on an upper surface of the substrate, which may be a PCB substrate, so as to be in direct contact with a fluid of thermocycling chamber 204, with an inner or lower serpentine trace embedded into, for example, a dielectric layer of the substrate. Thus, the heater may comprise one, two, or more than two, for example at least two serpentine traces. The at least two serpentine traces may be oriented perpendicular to each other and be provided in different layers or planes of substrate 204. The heater may comprise at least two perpendicularly oriented serpentine traces provided in different layers or planes of the microfluidic cartridge.

In some examples, the footprint of the heater extends beyond the footprint of thermocycling chamber 204, to ensure that any thermal edge effects are avoided and to ensure uniform heating in thermocycling chamber 204. As used herein, the term “footprint” refers to the area of the microfluidic device covered by, taken up or occupied by the component in question, corresponding to the physical layout of the component, for example in the X-Y plane or horizontal dimension. Thus, the “footprint” of the heater corresponds to the maximum dimensions of the heater, for example provided for by the maximum length and the maximum width of a serpentine trace. The footprint of the thermocycling chamber may therefore correspond to the area provided by the internal length and width of the thermocycling chamber. The heater footprint may be at least 5% greater than the footprint of thermocycling chamber 204, for example at least 10% greater, for example at least 15% greater, for example at least 20% greater, for example at least 25% greater, for example at least 30% greater, for example at least 40% greater, for example at least 50% greater. The heater may have an area, or a footprint, of from 50 mm² to 600 mm², for example from 60 mm² to 550 mm², for example from 70 mm² to 500 mm², for example from 75 mm² to 475 mm², for example from 80 mm² to 460 mm². The heater footprint dimension may be determined by the size of the thermocycling chamber to be heated, and may in some instances be dimensioned so as to have an overall footprint of 84 mm² (such as a 6×14 mm heater), or to have an overall footprint of 450 mm² (such as a 15×30 mm heater).

Regardless of configuration, the heater may receive electrical power from electrically conductive wires provided on or to the microfluidic cartridge to form an electrical circuit which supplies electrical current to the heater. Such components may be controlled by a controller located on or off the microfluidic cartridge via control signals. Heater configurations as described above enable temperature ramp rates of from 20 to 200° C. per second.

A dielectric layer may be disposed over the substrate and/or the heater embedded in or on the substrate. The dielectric layer may be spun on or sputtered onto the substrate. Thus, in some examples, the substrate comprises a dielectric coating of polyimide, SU-8, silicon oxide, silicon nitride, aluminium oxide, aluminium nitride or any combination/stack thereof. Another suitable material is Kapton®, which may be incorporated into a coating or stack with any of the aforementioned materials.

Thermocycling chamber 204 can be provided in a microfluidic layer (also termed a microfluidic stack) of microfluidic cartridge 201, disposed on the substrate. As used herein, the terms “microfluidic layer”, “microfluidic stack”, “fluidic layer” or “fluidic stack” refer to the components of the microfluidic cartridge through which one or more fluids can pass during use of the microfluidic cartridge, for example through one or more microfluidic channels and chambers. The terms are intended to encompass multiple flow paths, for example in different levels of the layer/stack, and distinguish these flow channel-containing components from other operational modules such as electronic circuitry and sensors. The microfluidic layer or microfluidic stack may comprise any material or combination of materials suitable for use in microfluidic cartridges, including polycarbonate, and cyclic olefin copolymer (COC). In some examples, thermocycling chamber 204 is formed in a microfluidic layer or microfluidic stack by moulding, or selectively etching or machining away regions of material so as to form a thermocycling chamber. In some examples, thermocycling chamber 204 is formed wholly within the material forming the microfluidic layer (for example COC), with that material also forming the base of thermocycling chamber 204. In other examples, the material forming the microfluidic layer forms the walls of thermocycling chamber 204, with the underlying substrate (for example with a dielectric layer as described) forming the base or floor of thermocycling chamber 204. In other examples, thermocycling chamber may be provided in one or more layers of pressure-sensitive adhesive that bonds the upper layers of the microfluidic stack to the substrate, with the upper surface of the substrate forming the base or floor of thermocycling chamber 204. Other layers present in a microfluidic stack may include layers of adhesive to bond the microfluidic layer to the substrate and/or bond layers of a microfluidic stack to each other. Suitable adhesives include pressure-sensitive adhesives, which can comprise an elastomer based on acrylic, silicone or rubber optionally compounded with a tackifier such as a rosin ester. Convenient pressure sensitive adhesives are in the form of double-sided films or tape, such as the acrylic adhesives 200MP and 7956MP available from 3M™. The microfluidic stack may include a transparent lid. The lid may be transparent in the visible region of the electromagnetic spectrum, and have low levels of autofluorescence to allow detection of fluorescence in thermocycling chamber 204. The lid may be formed from any suitable material, for example a thermoplastic material such as polycarbonate or COC as described above.

Each sample droplet generator is operable, or configured, to partition a PCR mixture into a plurality of aqueous droplets dispersed in a carrier liquid, and at least one sample droplet generator is operable, or configured to partition a PCR mixture that is different to a PCR mixture that is partitioned by at least one other sample droplet generator. As used herein, a “droplet generator” is operable or configured to partition a liquid into a plurality of aqueous droplets dispersed in a carrier liquid by directing a flow of that liquid into a stream of carrier liquid so as to produce a mono-disperse population of aqueous droplets of defined dimension in a reproducible manner. As used herein, a mono-disperse population of aqueous droplets is considered to contain droplets of uniform size, that is a population with a narrow droplet size distribution, for example deviating from a mean droplet diameter by less than 0.3%. The carrier fluid can be an oil, such as a hydrocarbon oil, and so the mono-disperse population may be termed a water-in-oil emulsion as the water-based aqueous droplets are dispersed in the oil. As used herein, a “sample droplet generator” is any fluid ejection device which is operatively coupled to a source of a PCR mixture, i.e. a mixture including a sample suspected of containing a target nucleic acid of interest and which is configured to eject an aqueous solution of the PCR mixture as a stream of droplets, as a dispersed phase, into a continuous phase of carrier oil. Droplet generators fall into two categories: passive systems, and active systems. The simplest form of droplet generator is a passive system based on cross-flow at a junction of two flow channels, with a first channel filled with the carrier liquid and the second channel introducing droplets of a desirable or defined size into the flowing carrier liquid. The junction may be arranged so that the second channel is perpendicularto the first channel, or substantially perpendicular, orthe second channel may meet the first channel at an acute or oblique angle. As the aqueous solution exits the second channel, droplets form by shearing off into the continuous oil phase at the junction between the two channels. More complex droplet generators include flow focusing systems, for example co-focusing systems in which the second channel is completely within the first channel, functioning as a nozzle, or cross-flow systems in which a flow channel is provided with a wall having a plurality of defined apertures or pores, through which the liquid to be dispersed can be injected from a second channel opposite the first channel. Further examples of droplet generators include active systems including nozzles, as well as devices based on those used in inkjet printhead assemblies, such as fluid ejection dies (which may comprise one or more nozzles), firing resistors, and piezo-electric and thermal inkjet ejectors. The sample droplet generator may be selected from a cross-flow junction between two flow channels, a nozzle projecting into a flow channel, and a fluid ejection die based on a piezo-electric or thermal inkjet ejector.

Droplet dimensions can be controlled by varying the dimensions of the respective channels (e.g. the diameter of the flow channel for a carrier fluid and the diameter of the flow channel for the PCR mixture), as well as the rate of flow of both carrier liquid and the liquid from which the droplets form. For example, a faster-flowing carrier liquid will cause a forming droplet to shear off as a droplet of smaller dimensions than if the carrier liquid was not flowing as fast. Suitable flow rates for the carrier fluid may be in the range of from 70 nL/min to 3 mL/min, for example from 100 nL/min to 2 mL/min, for example from 500 nL/min to 1 mL/min, for example from 1 μL/min to 500 μL/min, for example from 10 μL/min to 250 μL/min, for example from 100 μL/min to 200 μL/min. Suitable flow rates for the liquid to be dispersed (e.g. a PCR mixture) may be in the range of from 70 nL/min to 3 mL/min, for example from 100 nL/min to 2 mL/min, for example from 500 nL/min to 1 mL/min, for example from 1 μL/min to 500 μL/min, for example from 10 μL/min to 250 μL/min, for example from 100 μL/min to 200 μL/min. Under these conditions, a droplet generator having an aperture in the range of from 5 μm to 100 μm, for example from 10 μm to 50 μm, for example from 15 μm to 35 μm can produce mono-disperse populations from a starting volume of 20 μL of liquid (e.g. a PCR mixture) of up to 20,000 droplets having an average diameter (as determined optically using microscopy imaging) in the range of from 5 μm to 200 μm, for example from 10 μm to 150 μm, for example from 15 μm to 100 μm, for example from 20 μm to 50 μm. In some examples, each sample droplet generator is configured to disperse the plurality of aqueous droplets into the carrier fluid in a flow channel of the microfluidic cartridge, such that each droplet has a diameter at least 50% of the diameter of the flow channel, for example at least 60%, for example at least 70%, for example at least 80%, for example at least 90%. In this way, each droplet occupies substantially the entire width or diameter of the flow channel so that droplets proceed through the flow channel one after the other, increasing control of the multiplexing of the system.

As noted above, each sample droplet generator is operable, or configured, to partition a PCR mixture into a plurality of aqueous droplets dispersed in a carrier liquid, and at least one sample droplet generator is operable, or configured to partition a PCR mixture that is different to a PCR mixture that is partitioned by at least one other sample droplet generator.

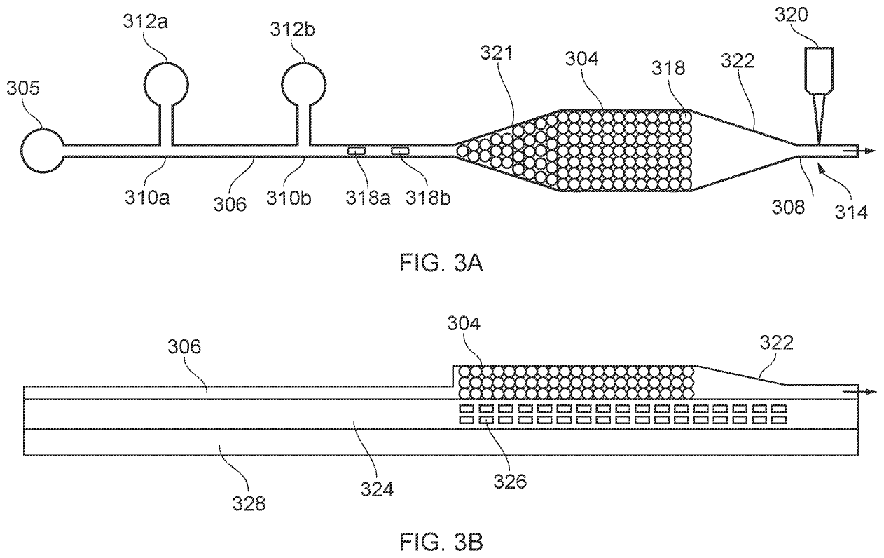

As used herein, a “PCR mixture” comprises a nucleic acid sample, which may contain a target nucleic acid, at least one pair of oligonucleotide primers designed to bind to and amplify the target nucleic acid, a chain elongating enzyme such as a DNA polymerase, dNTPs and any necessary co-factors. Once dispersed, each PCR mixture may be termed a droplet dispersion, or a PCR dispersion, which will be understood as including the plurality of aqueous droplets rather than a single dispersed droplet. In this way, a multiplexed ddPCR amplification is enabled in a single thermocycling chamber, as will now be described with reference to FIGS. 3 to 7.

FIGS. 3A and 3B show in plan view and in cross-section a microfluidic network of a microfluidic cartridge of the present disclosure. In FIG. 3A, a reservoir 305 for carrier liquid, for example a carrier oil, can be seen at one end of flow channel 306, which at its other end connects with an inlet to thermocycling chamber 304. The carrier liquid may be any liquid capable of stably dispersing the aqueous droplets, for example as a water-in-oil emulsion. The carrier liquid may be any suitable oil, or other hydrophobic liquid. Examples of suitable oils include hydrocarbon oils, including white mineral oils used for PCR purposes, such as those used to prevent evaporation of aqueous systems during amplification. Positioned along flow channel 306 between reservoir 305 and thermocycling chamber 304 are two sample droplet generators 310a and 310b, which in this image are junctions, in particular T-junctions, between flow channel 306 and side channels leading to sample reservoirs 312a and 312b. In FIG. 3A, thermocycling chamber 304 includes an inlet 321 opening out into thermocycling chamber and an outlet 322 which tapers toward the optical readout zone.

Thermocycling chamber 304 is also populated with a plurality of droplets 318, filling up the chamber, dispersed in a carrier fluid (not shown). Flow channel 306 also shows droplets 318a and 318b being transported in sequence, with the droplet size corresponding to the dimension of the flow channel. By controlling flow channel dimension and droplet size so that each droplet has a diameter approximately equal to but less than the diameter of the flow channel, controlled filling of thermocycling chamber can be achieved. As also seen in FIG. 3A, thermocycling chamber 304 has an outlet leading to flow channel 308. Optical sensor 320 is arranged so that it focuses on optical readout zone 314 of flow channel 308. Due to the tapering or narrowing of outlet 322 of thermocycling chamber 304, and the dimensions of flow channel 308, droplets 318 exit thermocycling chamber 304 individually, one after the other. In this way, only one droplet at a time enters into optical readout zone 314 and so the droplets can be interrogated optically individually as they exit thermocycling chamber 304.

FIG. 3B shows the cross-sectional view of FIG. 3A. Flow channel 306 is shown as being disposed on a substrate 324, in which a heater in the form of a plurality of heater elements 326 is embedded. Substrate 324 is also shown as being in direct contact with a cooling module 328, which can extract heat from substrate 324 and thermocycling chamber 304 during amplification. In the particular configuration shown, heater elements 326 extend across the width of thermocycling chamber 304, though other arrangements are possible. Each heater element 326 may be joined at one or both ends to a neighbouring or adjacent heater element to form, for example a comb-like heater or a serpentine heater. Two layers of heater elements are shown, both fully embedded within substrate 324, though as described elsewhere, an upper heater may be only partially embedded within substrate 324 but with an upper surface exposed to thermocycling chamber 304 so as to be in direct contact with a liquid contained within thermocycling chamber 304. In one example, as shown in FIG. 3B, thermocycling chamber 304 has a height greater than the height of flow channel 306 and flow channel 308, thus accommodating a greater number of dispersed droplets with droplets being stacked in the vertical as well as the horizontal direction. In this way, a greater number of droplets can be thermocycled at any one time, increasing sensitivity and output of the system. In addition to being tapered in a horizontal direction, outlet 322 of thermocycling chamber 304 tapers or narrows in the vertical direction as can be seen in FIG. 3B, with the height of thermocycling chamber 304 becoming shorter in the vicinity of the outlet to flow channel 308. This vertical tapering, in combination with the horizontal tapering, ensure that only one droplet at a time enters into optical readout zone 314 and so the droplets can be interrogated optically individually as they exit thermocycling chamber 304.

In use, each of sample reservoirs 312a and 312b can be filled with a different PCR mixture. For example, a first PCR mixture of a sample with a first PCR Master Mix can be introduced into sample reservoir 312a, while a second PCR mixture of the same sample with a second, and different PCR Master Mix can be introduced into sample reservoir 312b. First and second PCR Master Mixes may differ in the nature of the PCR primer pairs, for example, and/or in the nature of a reporter molecule. Having different primer pairs enables the same sample to be interrogated for the presence of different target nucleic acids in the same multiplexed reaction, while having different reporter molecules enables each droplet to be differentiated from other droplets based on optical output. For example, a first PCR Master Mix may be provided with SYBR Green I, which fluoresces with a λₘₐₓ of 520 nm, while a second PCR Master Mix may be provided with a Cy5 reporter, which has a fluorescence emission at 670 nm. Other reporter dyes include the Alexa Fluor series of dyes, which have emission wavelengths ranging from 440 nm to 805 nm. Thus, different PCR Master Mixes with different primer pairs and different reporter molecules can be prepared and mixed with aliquots of sample nucleic acid to form different PCR mixtures, which can be introduced into sample reservoirs 312a and 312b. In the example in which a sample is to be amplified with different PCR Master Mixes (for example different primer pairs) the different PCR Master Mixes can be pre-prepared, and introduced into their respective sample reservoirs in lyophilized form on a surface of the reservoir, or they can be contained (in solid or solution form) within a frangible package, for example a blister pack, within the sample reservoir 312a/312b. Since each sample droplet generator in FIG. 3A is coupled to its own sample reservoir containing a unique PCR Master Mix or a unique PCR mixture, each sample droplet generator is operable to partition a PCR mixture that is different to a PCR mixture that is partitioned by the other sample droplet generator.

Once sample reservoirs 312a and 312b have been filled with their respective PCR mixtures, a carrier fluid can be pumped from reservoir 305 along flow channel 306, while a PCR mixture present in sample reservoir 312a can be pumped to sample droplet generator 310a so as to partition the PCR mixture into a plurality of aqueous droplets dispersed in the carrier liquid, which are then pumped into thermocycling chamber 304. Subsequently, the PCR mixture present in sample reservoir 312b (which is different to the PCR mixture from sample reservoir 312a now present in thermocycling chamber 304 as the droplet dispersion) can be pumped to sample droplet generator 310b so as to partition that different PCR mixture into a plurality of aqueous droplets dispersed in the carrier liquid, which are then pumped into thermocycling chamber 304. As used herein, the term “droplet dispersion” refers to a water-in-oil emulsion produced by a droplet generator partitioning a PCR reaction mixture (containing sample nucleic acid, polymerase, PCR primers, dNTPs and co-factors) into a plurality of droplets dispersed in a carrier liquid. Each droplet is a self-contained PCR amplification system, separated from the others by the carrier liquid.

After thermocycling has completed, the droplets are pumped from thermocycling chamber 304 into flow channel 308 and into optical readout zone 314. After each droplet has passed through optical readout zone 314, it continues along flow channel 308 to a waste reservoir. With each droplet statistically expected to contain no more than about one copy number of target nucleic acid, and with different primers and reporter molecules being used, a simple droplet count of fluorescent droplets and non-fluorescent droplets for each sample-Master Mix combination allows a highly sensitive and quantitative readout of the digital droplet PCR amplification. While FIGS. 3A and 3B show two sample reservoirs and two sample droplet generators, other configurations include three, four, five or more sample reservoirs each coupled to a separate sample droplet generator, so that even higher degrees of multiplexing can be accomplished.

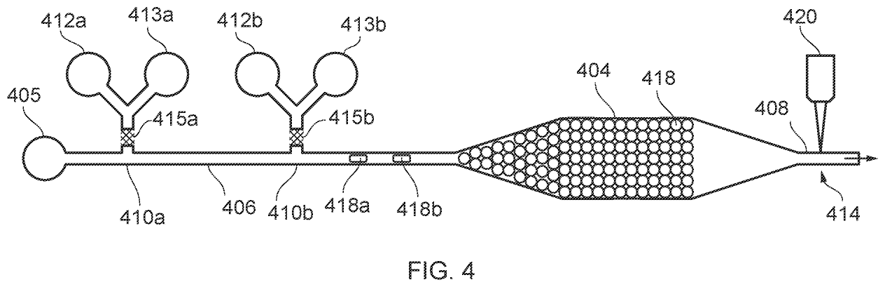

FIG. 4 shows an alternative configuration to that shown in FIG. 3A, with each sample droplet generator fluidly connected to a respective sample reservoir and a respective reagent reservoir. As with the configuration of FIG. 3A, FIG. 4 shows a carrier fluid reservoir 405 opening out into a flow channel 406, which leads to thermocycling chamber 404. Thermocycling chamber 404 opens out into flow channel 408 with optical readout zone 414 at the focal point for optical sensor 420. The configuration of FIG. 4 differs in the nature of the sample reservoirs. In FIG. 4, a plurality of sample reservoirs 412a and 412b are shown, each fluidly connected to a dedicated reagent reservoir 413a and 413b respectively. Each reagent reservoir 413a and 413b is operatively coupled to at least one sample droplet generator, and can be provided with a different PCR Master Mix. The PCR Master Mix can be in dry form on a surface of a reagent reservoir or in a frangible package such as blister pack as described above. Aliquots of a sample can be introduced into sample reservoir 412a and sample reservoir 412b. Each aliquot can then be pumped toward flow channel 406, concurrently with its respective PCT Master Mix from the respective reservoir. The two solutions from reservoirs 412a and 413a meet at mixer 415a, to be combined and form a PCR mixture containing sample suspected of containing a target nucleic acid, and the reagents for a PCR amplification. Microfluidic mixers function by increasing turbulent flow within a chamber, and can be passive or active mixers. Active mixers may involve the use of sonication, magnetic stirring, and bubble-induced acoustic actuation, while passive mixers may be based on multiple splitting, recombining, and rotating channels, creating turbulent flow paths that cause the two liquids to mix. Once mixer 415a has combined the sample and PCR Master Mix to form a PCR mixture, this mixture is then partitioned into a droplet dispersion in a carrier liquid by sample droplet generator 410a, with the produced dispersion being pumped into thermocycling chamber 404. In a similar manner, a second droplet dispersion can be produced by sample droplet generator 410b, located downstream from mixer 415b. and served by sample reservoir 412b and reagent reservoir 413b. As described above for FIG. 3, since each sample droplet generator in FIG. 4 is coupled to a sample reservoir and a reagent reservoir containing a unique PCR Master Mix, each sample droplet generator is operable to partition a PCR mixture that is different to a PCR mixture that is partitioned by the other sample droplet generator, with the difference being that sample and PCR reagents are separately pumped to a microfluidic mixer and mixed immediately prior to reaching the sample droplet generator. As with FIG. 3, droplets 418a and 418b are pumped along flow channel to thermocycling chamber 404, in which the plurality of droplets 418 are thermocycled before being pumped out the chamber to optical readout zone 414 in flow channel 420 to be read by optical sensor 420.

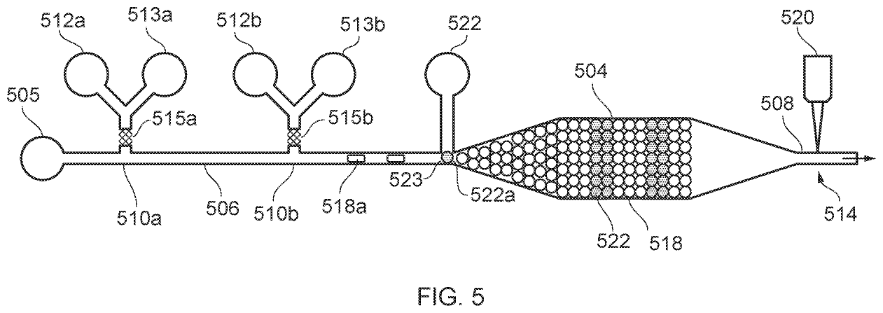

FIG. 5 shows yet another configuration, which enables even higher degrees of multiplexing within a digital droplet PCR amplification in which each sample droplet generator is fluidly connected to a respective sample reservoir and a common reagent reservoir which is fluidly connected to at least one other sample droplet generator. FIG. 5 shows the same arrangement of components as that shown in FIG. 4, with a carrier fluid reservoir 505 opening out into a flow channel 506, which leads to thermocycling chamber 504. Thermocycling chamber 504 opens out into flow channel 508 with optical readout zone 514 at the focal point for optical sensor 520. Sample reservoirs 512a and 512b are paired respectively with reagent reservoirs 513a and 513b, and mixers 515a and 515b are located so as to mix incoming sample and PCR Master Mix reagents into a single PCR mixture which can be partitioned by sample droplet generators 510a and 510b respectively.

The configuration of FIG. 5 includes a separator dye reservoir 522, which is fluidly connected to a separator droplet generator 523 at a junction with flow channel 506. Separator reservoir 522 can be filled with a separator dye liquid or solution, or with some other marker molecule, which can be partitioned and dispersed into a carrier liquid by separator droplet generator 523 so as to produce a dispersion of dye droplets 522, with an example droplet 522a shown leaving separator droplet generator 523. As can be seen in thermocycling chamber 504, timed pumping of PCR mixtures and separator solutions can result in a dispersion of separator droplets 522 acting as a divider or separator between groups of PCR reaction droplets 518. In this way, different droplet dispersions based on different samples, or different PCR Master Mixes, can be introduced into a single thermocycling chamber and amplified together in parallel. As with the earlier described Figures, each droplet is optically interrogated when passing through optical readout zone 514. However, through the use of suitable separator dyes, as is described later in connection with the method of the present disclosure, optical sensor 520 can be configured to detect whether each droplet is (i) fluorescent, by virtue of a successful amplification of a target nucleic acid; (ii) non-fluorescent, by virtue of an unsuccessful amplification due to the absence of a target nucleic acid; or (iii) a separator droplet, by virtue of the separator dye being detected by the optical sensor. The use of the separator droplets with a dye reporter that is not used as an amplification reporter enables a computer control module controlling the PCR system to identify the start and end of each particular droplet dispersion.

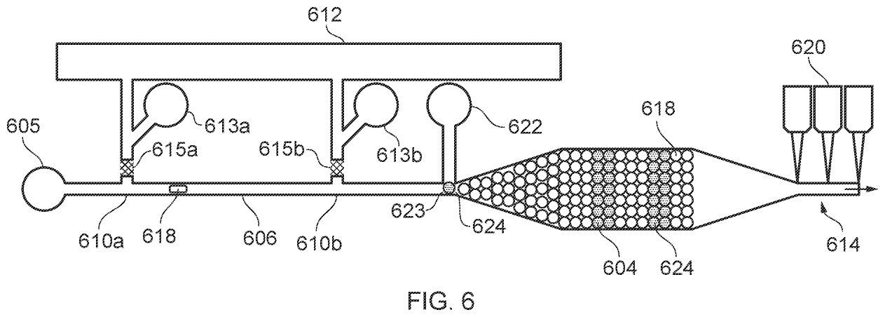

FIG. 6 shows yet another configuration in which each sample droplet generator is fluidly connected to a respective reagent reservoir and a common sample reservoir which is fluidly connected to at least one other sample droplet generator. FIG. 6 shows a similar arrangement of components to that shown in FIG. 5, with a carrier fluid reservoir 605 opening out into a flow channel 606, which leads to thermocycling chamber 604. Thermocycling chamber 604 opens out into flow channel 608 with optical readout zone 614 at the focal point for optical sensor 620. In this configuration, optical sensor 620 is shown as a multi-wavelength fluorimeter, capable of exciting fluorophores of different wavelengths and detecting their respective and unique fluorescent emissions. The configuration of FIG. 6 also includes a separator reservoir 622, which is in fluid communication with a separator droplet generator 623 at a junction with flow channel 606 to produce separator droplets 624. In this configuration, instead of the sample reservoirs being paired with PCR Master Mix or reagent reservoir on a 1:1 basis, a single sample reservoir 612 is provided. Sample reservoir 612 has two outlets (not numbered), coupling it to two separate PCR Master Mix reagent reservoirs 613a and 613b. Sample and PCR Master Mix from reservoir 613a are combined in mixer 615a, while the same sample from sample reservoir can be combined with a different PCR Master Mix from reservoir 613b by mixer 615b. Thus, through the use of a common sample reservoir and separate reagent reservoirs each primed with different Master Mixes, each sample droplet generator is not only operable to partition a PCR mixture of sample and Master Mix into a plurality of aqueous droplets 618 dispersed in a carrier liquid, but is also operable to partition a PCR mixture that is different to a PCR mixture that is partitioned the other sample droplet generator. In this way, a single sample can be tested for different target nucleic acids in a multiplexed ddPCR system.

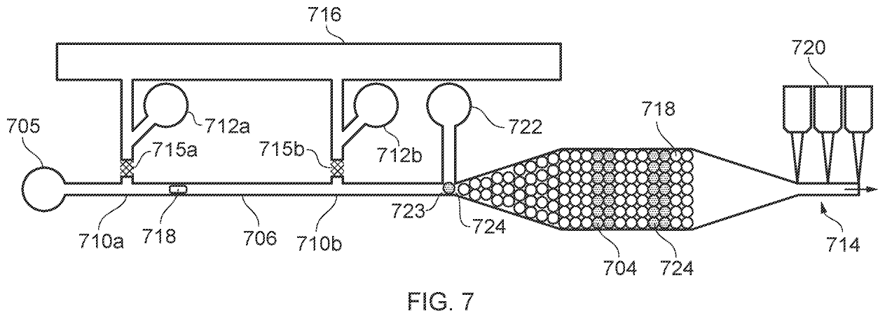

FIG. 7 shows yet another configuration. FIG. 7 shows a similar arrangement of components to that shown in FIG. 6, with a carrier fluid reservoir 705 opening out into a flow channel 706, which leads to thermocycling chamber 704. Thermocycling chamber 704 opens out into flow channel 708 with optical readout zone 714 at the focal point for optical sensor 720. The configuration of FIG. 7 also includes a separator reservoir 722, which is in fluid communication with a separator droplet generator 723 at a junction with flow channel 706 to produce separator droplets 724. In this configuration, instead of a single sample reservoir being operatively coupled to a separator reagent reservoirs, a single reagent reservoir 716 is provided. Reagent reservoir 716 has two outlets (not numbered), coupling it to two separate sample reservoirs 712a and 712b. A PCR Master Mix from reagent reservoir 716 and sample from reservoir 712a are combined in mixer 715a, while a different sample from sample reservoir 712b can be combined with the PCR Master Mix from reservoir 716 by mixer 715b. Thus, through the use of a common reagent reservoir and separate sample reservoirs, each sample droplet generator is not only operable to partition a PCR mixture of sample and Master Mix into a plurality of aqueous droplets 718 dispersed in a carrier liquid, but is also operable to partition a PCR mixture that is different to a PCR mixture that is partitioned the other sample droplet generator. In this way, different samples can be introduced into each sample reservoir, enabling rapid thermocycling and testing of different samples under the same conditions in a multiplexed ddPCR system.

Thermocycling chamber 204 may have an internal volume of less than 100 μL, for example less than 75 μL, for example less than 50 μL, for example less than 30 μL, for example less than 20 μL, for example about 15 μL, depending on configuration.

Thermocycling chamber 204 may also be referred to as a flow cell, and is may be dimensioned so as to have an aspect ratio of its largest dimension, for example in the XY plane, to its height of from 10:1 to 1000:1, for example from 50:1 to 1000:1, for example from 100:1 to 1000:1, for example from 500:1 to 1000:1. In some examples, the aspect ratio may be from 10:1 to 900:1, for example from 10:1 to 750:1, for example from 10:1 to 500:1, for example from 10:1 to 250:1, for example from 10:1 to 150:1. Thinner fluid thicknesses allow for a more rapid, and more uniform heat transfer from an embedded heater in microfluidic cartridge 201, and a subsequent more rapid, and more uniform heat transfer from the fluid to a cooling module. In some examples, thermocycling chamber 204 has a height of less than 500 μm, for example less than 400 μm, for example less than 300 μm, for example less than 200 μm, for example a height of from 500 μm to 50 μm, for example a height of from 400 μm to 75 μm, for example a height of from 300 μm to 100 μm, for example a height of from 200 μm to 100 μm, for example a height of about 150 μm to 200 μm.

In some examples, the microfluidic layer is provided with one or more fluid inlets and outlets to provide a liquid such as a reaction liquid to the or each thermocycling chamber. In some examples, the outlet may also be referred to as a vent. The presence of a vent enables unhindered flow of liquid through the thermocycling chamber and minimises risk of unwanted bubble formation within the thermocycling chamber. The outlet or vent may be provided with a membrane. The membrane may be a gas-permeable membrane, a flexible membrane, a rupturable or non-rupturable membrane or a membrane which exhibits one or more of these properties. The membrane may be formed of a hydrophobic material, or a hydrophilic material.

In some examples, the or each thermocycling chamber is also provided with an overflow chamber, which may be a dedicated overflow chamber or an overflow chamber common to all thermocycling chambers. The overflow chamber may be provided in a different layer of the microfluidic stack and may be fluidly connected to waste reservoir 216. Depending on use, waste reservoir 216 and/or the overflow chamber may be provided with a permeable or non-permeable, or rupturable or non-rupturable seal. In particular configurations of the microfluidic cartridge, an overflow chamber may be associated with a thermocycling chamber, with a capillary break located at the inlet to the overflow chamber and configured to control flow of incoming liquid into the thermocycling chamber and only permitting liquid to enter the overflow chamber once the thermocycling chamber is filled. As can be seen in FIG. 2, an overflow chamber 212 is provided in an upper layer of the fluidic stack, and is connected to flow channel 206 via an inlet 211 which is constricted (for example by having a narrower diameter or dimension) relative to flow channel 206. Due to capillary forces, the pinhole inlet 211 serves as a capillary break, preventing fluid flow into overflow chamber 212 and instead directing flow of infilling liquid into thermocycling chamber 204 until thermocycling chamber 204 is full.

Thermocycling chamber 204 may be provided with at least one capillary break on an internal surface of the thermocycling chamber. The at least one capillary break controls the location of the liquid-gas interface during filling to avoid bubble formation.

In some examples, the thermocycling chamber has a high aspect ratio of at least 1:10 (height to width) and is provided with at least one capillary break on an internal surface of the thermocycling chamber, to control the location of the liquid-gas interface during filling to avoid bubble formation. In some examples, the at least one capillary break comprises a region of raised material on, or depressed material in, the floor or ceiling of the chamber. In some examples, the at least one capillary break can be a region of the floor or ceiling of the thermocycling chamber with a different contact angle to the contact angle of the surrounding floor or ceiling, for example by depositing or printing a material having a higher hydrophobicity than the surrounding material. For example, the at least one capillary break may be formed by depositing at pre-defined locations a hydrophobic material onto a dielectric layer. The dielectric layer and the hydrophobic material may be as described. In some examples, the at least one capillary break comprises a resistor.

In some examples, the thermocycling chamber is provided with one or more thermally controllable capillary breaks on an internal surface thereof. In some examples, the at least one capillary break is thermally activated or controllable, thereby providing control over when an infilling liquid is able to pass or burst the at least one capillary pressure barrier. In some examples, the at least one capillary break is thermally activated via optical means, for example via selective absorption of radiation from an optical source. For example, the at least one capillary break may be formed of a material that selectively absorbs light of a particular wavelength and thereby generates heat. Selective absorber materials include ceramic, or metal oxide, materials such as copper oxide or cobalt oxide deposited on a substrate. In some examples, when the at least one capillary break comprises an electrode assembly (for example a plurality of printed electrical traces) the at least one capillary break is thermally activated by providing an electrical current to the electrode assembly. In some examples, the at least one capillary break comprises an electrode assembly, for example a printed electrode on a PCB substrate.

In operation, the liquid volume enters the thermocycling chamber and the liquid-gas interface is pinned at a first capillary break (for example a first electrical trace). A short thermal pulse quickly raises the temperature locally to the capillary break at this interface (but negligibly elsewhere), locally reducing the surface tension, and so allowing the interface to unpin. The unpinning may initially occur at a particular section of the capillary break and then propagate along the length of the capillary break. The preferential unpinning at a location may be controlled by a lower difference in surface tension or a lower step height of the capillary break. Such a mode of unpinning may be used to control how the liquid fills the region between sequential capillary breaks.

The liquid volume then proceeds by capillary action to further fill the chamber, until the next trace. The next pulse then unpins the interface there and the process repeats. The pulses are timed to allow the fluid to fill the region between the traces.

In some examples, the thermocycling chamber is provided with a plurality of capillary breaks on the floor of the thermocycling chamber. In some examples, the thermocycling chamber is provided with a plurality of capillary breaks on the ceiling of the thermocycling chamber. In some examples, the thermocycling chamber is provided with a plurality of capillary breaks on the floor and the ceiling of the thermocycling chamber. In some examples, the plurality of capillary breaks on the floor and the ceiling of the thermocycling chamber are aligned opposite to each other, or in an alternating pattern. In some examples, the plurality of capillary breaks are independently or commonly activated by electrical heating, or by optical heating as described herein.

In some examples, the electrode assembly performs multiple functions, including thermal control of the filling of the thermocycling chamber, and thermal control of the liquid volume during a thermocycling reaction as described later in connection with the methods of the present disclosure.

In some examples, the microfluidic cartridge is in the form of a cassette, or chip, to be used in the PCR system. In some examples, the microfluidic cartridge may be a single use or disposable cartridge. In some examples, the microfluidic cartridge may be configured to be inserted into or received by a port in system. In some examples, the microfluidic cartridge may be provided with one or more fluidic connections that are configured to engage with one or more corresponding fluidic connections in system, to enable fluid flow from the system into the microfluidic cartridge, for example to enable transfer of a sample injected into an injection port of the system to be transferred to a thermocycling chamber of the microfluidic cartridge. In other examples, the or each thermocycling chamber of the microfluidic cartridge may be filled with sample prior to inserting the microfluidic cartridge into the system, for example by manual pipetting a sample solution through an inlet port such as a Luer connector or membrane valve.

### Pressure-Actuated Pump

PCR system 100 is provided with a pressure-actuated pump 102, also referred to as a pressure driven fluid driver. The pressure-actuated pump may be, for example, one or more hydraulic pumps coupled to a flow channel inlet and which can build back-pressure in a system to drive fluid through a channel. While other forms of pumping fluids through a microfluidic system are known, a pressure-actuated pump is best suited as thermally actuated systems which cause fluid flow by transient cavitation of air bubbles may disturb the droplet dispersions and cause droplets to coalesce together. Pressure pumps can be single channel or multi-channel, and can be coupled to flow sensors to control flow of each component out of a reservoir and into a flow channel with high levels of precision in terms of start and finish timings of one channel relative to another, and volumes ejected from reservoirs. Suitable pressure driven fluid drivers include the 4- or 8-channel MFCS™ microfluidic flow controllers from Fluigent®.

### Cooling Module

PCR system can be provided with a cooling module 103 to enable rapid and efficient cooling of a PCR mixture undergoing amplification in a thermocycling chamber of the microfluidic cartridge. Cooling module 103 may be any suitable cooling module that when in thermal contact with microfluidic cartridge 101 enables good thermal transfer from microfluidic cartridge 101. As has been described above, the heater of the microfluidic device provides heat to the thermocycling chamber in order to reach denaturing conditions of a PCR mixture. In order to rapidly cool the PCR reaction mixture to an annealing temperature, rapid heat transfer away from the PCR mixture through thermal contact between the microfluidic device and the cooling module is required. As used herein, two components (for example a cooling module and a microfluidic cartridge) are in thermal contact with one another if heat transfer occurs when one component is at a higher temperature than the other component. Efficient thermal transfer can be achieved by having cooling module 103 in direct (e.g. physical) contact with microfluidic cartridge 101 for conductive cooling, although cooling modules that cool microfluidic cartridge 101 via convective means are also suited. It will be understood that “direct” contact with microfluidic cartridge 101 does not exclude the presence of intermediate materials so long as these are also thermally conductive and enable, rather than prevent, heat transfer. Efficient thermal transfer can be increased by providing clamping means to clamp the microfluidic cartridge to the cooling module, to maintain good thermal contract. One example of a cooling module is a coolant circuit with associated refrigeration systems through which a working fluid is cycled, and heat pipes. Suitable working fluids include water, with other coolants or refrigerants including glycols (e.g. ethylene glycol or propylene glycol), which may be used alone or blended with water. In some examples, a layer of compliant thermal interface material is provided between cooling module 103 and microfluidic cartridge 101, though this layer is optional. If included, compliant thermal interface material may be any thermally conductive material that enables good thermal contact and transfer, such as indium and indium alloys, such as In—Ag; gallium and gallium alloys; copper, aluminium, and lead. Suitable materials also include greases or polymer suspensions of silver, carbon micro- and nano-particles, aluminium oxide, boron nitride, zinc oxide, aluminium nitride, where the polymer material can be epoxy-based, silicone-based, urethane-based, and/or acrylate-based.

Cooling module 103 may also be in the form of heat pipe, in which a working liquid (such as water or another coolant) flows along a porous liquid path on an internal surface of the pipe, for example by capillary flow. Heat is transferred from the microfluidic cartridge to the heat pipe, causing the working fluid to evaporate, with the vapor flowing counter-current to the liquid flow. As the vapor travels to the end of the heat pipe furthest from the region which contacts the microfluidic cartridge, it condenses on the inner surface of the heat pipe, aided for example by an associated heat sink, in the process releasing the latent heat that was absorbed from the microfluidic cartridge. Heat pipes can be constructed from copper for water-based systems, but can also be constructed from aluminium, steel, or other thermally conductive materials. While water has been mentioned as a working liquid for the heat pipe, other working liquids which can readily be vaporized and condensed include ammonia.

The cooling module may be configured based on the configuration of the microfluidic cartridge to be used. For example, if the microfliudic cartridge comprises a plurality of thermocycling chambers provided in at least two groups, the cooling module may comprise a number of cooling panels equivalent to the number of groups of thermocycling chambers. Having two cooling panels, each serving a group of thermocycling chambers together enables, for example, different heating and cooling protocols to be performed on a single microfluidic cartridge. Alternatively, a simplified cooling module can be obtained by connecting the cooling panels in series.

In use, cooling module 103 may be operated in thermal contact with microfluidic cartridge 101 and at a constant temperature of no more than 35° C., for example no more than about 30° C., to ensure cooling rates of 30° C. per second within the thermocycling chamber. Achieving such temperatures may include flowing water, propylene glycol or any other suitable coolant or working fluid through the cooling module at a rate of from 1 L/min up to 5 L/min, for example from 1.1 L/min to 4 L/min, for example from 1.3 L/min to 3.5 L/min, for example from 1.5 L/min to 3.3 L/min, for example from 2 L/min to 3 L/min.

### Optical Sensor

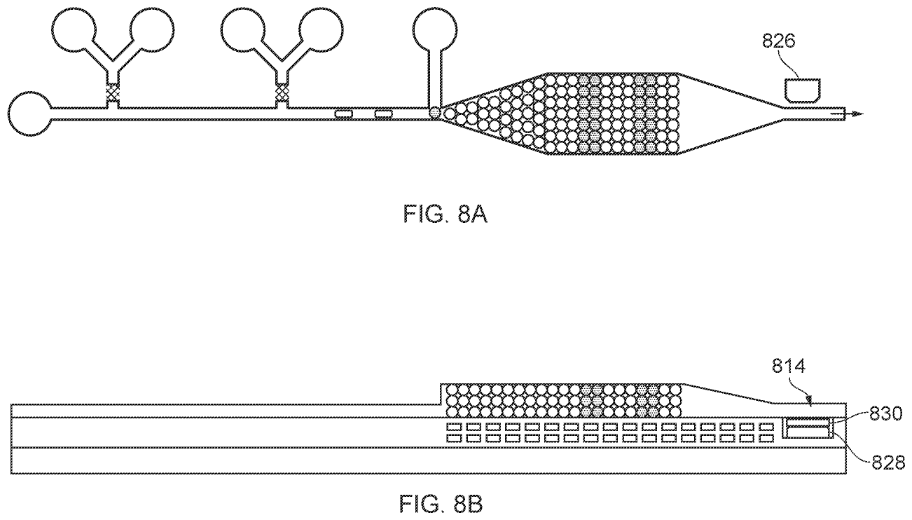

The PCR system 100 comprises an optical sensor configured to obtain optical signals from the optical readout zone of the microfluidic cartridge. In some examples, the optical sensor is a fluorescence sensor and the optical signals are fluorescence signals. As described above, fluorescent molecules are used as reporter molecules in PCR amplification, with the fluorescence intensity proportional to the amount of amplified nucleic acid material. In some examples, the optical sensor comprises a light source and a detector, wherein the light source is for example a laser diode, or an LED, configured to emit light of a wavelength suitable to cause fluorescence of a fluorescent reporter molecule during a PCR amplification process. For example, SYBR Green I, absorbs blue light with a λₘₐₓ of 497 nm, and emits green light with a λₘₐₓ of 520 nm. In some examples, the detector may be a charge coupled device (CCD) or pin photodiode configured to detect the emitted fluorescent light. In some examples, the detector may be a charge coupled device (CCD) or pin photodiode to detect the emitted fluorescent light. In some examples, the optical sensor is arranged above or below the thermocycling chamber, for example above or below a plane in which the liquid sample is being thermocycled. In some examples, microfluidic cartridge 101 is provided with an optical window or opening that allows transmission of light therethrough to an optical sensor located in PCR system 100 but external to microfluidic cartridge 101, or within microfluidic cartridge 101 itself. In some examples, the optical sensor is integrated or embedded into a lid or other component of microfluidic cartridge 101. FIGS. 8A and 8B show the coniguration of FIG. 5, including an example of an integrated optical sensor, such as an integrated fluorimeter. In FIG. 8A, an edge emitting LED as light emitter 826 is integrated into the microfluidic cartridge, with the emitted wavelength being selectable dependent on the nature of the fluorophore reporter molecule being used. FIG. 8B shows a corresponding photodetector 828 embedded into the PCB substrate, with an optical filter 830 between photodetector 828 and optical readout zone 814.

### Other Components

In some examples, PCR system 100 comprises an electrical interface, configured to contact an electrical interface provided on microfluidic cartridge 101. The electrical interface on microfluidic cartridge 101 may be coupled to any component of the cartridge that requires electrical current to operate. Examples of such devices include the heater elements, either in flat panel form or printed conductive trace form, and actuators for controlling fluid flow within the microfluidic cartridge. In some examples, the electrical interfaces may be multi-pin input/output off board connecters, for example 44-pin connectors that enable electrical coupling of the microfluidic cartridge to a computer module of the PCR system. Each pin of the electrical interface may provide an electrical contact to a specific component of the microfluidic cartridge, such as the individually addressable or controllable heaters described herein. The electrical coupling of the cartridge to the system allows control signals from the computer module to be sent to the cartridge so that electrical current can be sent to desired modules of the cartridge.

As noted above, PCR system 100 may comprise a computer control module. In some examples, the computer control module comprises a processor comprising hardware architecture to retrieve executable code from a data storage device or computer-readable medium and execute instructions in the form of the executable code. The processor may include a number of processor cores, an application specific integrated circuit (ASIC), field programmable gate array (FPGA) or other hardware structure to perform the functions disclosed herein. The executable code may, when executed by the processor, cause the processor to implement the functionality of one or more hardware components of the cartridge and/or system such as one or more heaters and/or one or more optical detectors. In the course of executing code, the processor may receive input from and provide output to a number of the hardware components, directly or indirectly. The computer control module may communicate with such components via a communication interface which may comprise electrical contact pads, electrical sockets, electrical pins or other interface structures. In one example, the communication interface may facilitate wireless communication.

In some examples, the computer control module initiates and controls the formation of droplet dispersions by instructing and controlling the pressure driven fluid driver to initiate flow of a carrier liquid (e.g. an oil) into a flow channel, and to initiate flow of a PCR mixture to a sample droplet generator. Depending on the ddPCR system being used, the computer control module may initiate and control the formation of droplet dispersions by instructing and controlling the pressure driven fluid driver to initiate flow of a separate sample and PCR Master Mix to bring these to a mixer before pumping the resulting PCR mixture to a sample droplet generator.
introduction of a sample into the thermocycling chamber, or into multiple thermocycling chambers. For example, the computer control module may control a series of valves and pumps in the system or on the microfluidic cartridge to direct flow of a dispersion of a test sample or solution to the thermocycling chamber.

In some examples, the computer control module may further control the processing of a sample in a thermocycling chamber, for example by subjecting the thermocycling chamber to thermocycling conditions. For example, the computer control module may control, through the output of control signals, the operation of one or more heaters to control the temperature and duration of heating within the or each thermocycling chamber, or the operation of one or more valves or pumps within a cooling module to control the temperature of the cooling module and thereby provide cooling to a reaction mixture provided in the thermocycling chamber. As a result, a sample may undergo various selected reactions, various selected heating cycles and various sensing operations under the control of the computer control module.

### Method of Performinq PCR

A method of performing PCR is described, comprising:
partitioning, in a microfluidic cartridge, a first PCR mixture into a plurality of aqueous droplets and dispersing the plurality of aqueous droplets in a carrier liquid to form a first PCR dispersion;
transporting the first PCR dispersion into a thermocycling chamber of the microfluidic cartridge;
partitioning, in the microfluidic cartridge, a second PCR mixture into a plurality of aqueous droplets and dispersing the plurality of aqueous droplets in a carrier liquid to form a second PCR dispersion;
transporting the second PCR dispersion into the thermocycling chamber to form a reaction emulsion of first and second PCR dispersions;
subjecting the reaction emulsion to conditions suitable for amplification using polymerase chain reaction; and
successively transporting each droplet of the reaction emulsion from the thermocycling chamber to an optical readout zone of the microfluidic cartridge and detecting an optical signal from each droplet.

The method may be performed on a microfluidic cartridge as described herein, or on a PCR system as described herein comprising the microfluidic cartridge described herein. In some examples, the microfluidic cartridge is coupled to a pressure-actuated pump configured to pump carrier liquid through a flow channel to the thermocycling chamber, and to respectively pump the first PCR mixture and the second PCR mixture to the flow channel so as to form the first and second PCR dispersions.

The first PCR mixture and the second PCR mixture may each comprise a sample suspected of containing a nucleic acid of interest, and a PCR Master Mix. In some examples, the first PCR mixture differs from the second PCR mixture by having a different nucleic acid sample for amplification, or a different PCR Master Mix, as will be described below.

A sample suspected of containing a nucleic acid of interest can be introduced into a microfluidic cartridge of a PCR system by any suitable means. For example, the sample may be directly introduced by manual injection using a pipette into a sample inlet port provided on the microfluidic cartridge, or indirectly by introducing the sample into a sample port of the PCR system which is external to the microfluidic cartridge, with the sample then being pumped into the microfluidic cartridge.

The sample may comprise a nucleic acid sample obtained from a subject. In some examples, the nucleic acid sample may comprise a nucleic acid for analysis and is to be amplified in a method as described herein. In some examples, the nucleic acid sample may comprise a plurality of nucleic acids for analysis which are to be amplified in a method as described herein. In some examples, the sample is suspected of comprising a one or a plurality of nucleic acid sequences of interest. In some examples, the nucleic acid sample is obtained from one or more of a blood sample, a tissue sample, a saliva sample or mucosal sample. In some examples, the nucleic acid sample is obtained using a swab. In some examples, the nucleic acid sample is isolated from the bodily fluid or tissue via which it was obtained. Isolating the nucleic acid ensures that no other component of the sample is present which could inhibit PCR amplification. In some examples, the isolated nucleic acid sample obtained from a subject is dissolved or dispersed in an aqueous solution prior to being introduced into the microfluidic cartridge.

A PCR “Master Mix” is also introduced into the microfluidic cartridge. A PCR Master Mix is an aqueous solution of PCR reagents, already at optimized concentrations, which can be readily aliquoted and added to the sample. The Master Mix usually comprises the DNA elongation enzyme (e.g. a polymerase), the dNTPs, MgCl₂ as an enzyme co-factor (although other co-factors, such as MgSO₄ may be used with certain enzymes), all dissolved in an aqueous buffer. Suitable polymerases include the thermostable polymerases Taq, Bst and Pfu. The Master Mix may also include a reporter molecule, such as a non-specific fluorescent dye, such as SYBR Green, which intercalate into any double-stranded DNA, leading to an increase in fluorescence as more double-stranded DNA is produced, while other suitable reporter molecules include target-specific fluorescent reporter molecules, such as the TaqMan hydrolysis probes. The reporter molecule may be dissolved in the Master Mix, or may be covalently bound to a primer. The LightCycler® 480 SYBR Green I Master Mix includes a polymerase, co-factor, dNTPs and SYBR Green I in a buffered solution, meaning that only the nucleic acid sample (and, if appropriate, a primer) need to be added. However, the reporter molecule may also be added separately.

Similarly to the sample, a PCR Master Mix may be introduced into the microfluidic cartridge in a number of ways. The PCR Master Mix may be introduced into a central reservoir, with aliquots being pumped to one or more sample droplet generators, or may be introduced into a specific reservoir, with aliquots being pumped to a single sample droplet generator. The PCR Master Mix may be provided in dried form, along with a frangible package, for example a blister pack, containing buffer which can be ruptured to release the buffer so as to dissolve the PCR Master Mix into a reagent reservoir from which it can be pumped as required. The PCR Master Mix may be provided in liquid form within a frangible package, for example a blister pack, which can be ruptured to release the PCR Master Mix into a reagent reservoir from which it can be pumped as required.

A PCR mixture comprising sample and PCR Master Mix to be partitioned may have a volume of less than 100 μL, for example less than 50 μL, for example less than 25 μL, for example less than 10 μL, for example about 5 μL. In some examples, the PCR mixture may have a volume of greater than 5 μL, for example greater than 10 μL, for example greater than 25 μL, for example greater than 50 μL, for example about 100 μL.

As described above, a multiplexed digital droplet PCR amplification of the present disclosure uses different droplet dispersions or PCR dispersions in which the PCR mixture within the droplets of a first PCR dispersion is different to the PCR mixture within the droplets of a second PCR dispersion by virtue of them containing different samples, or different primer pairs, for example. The discussion which follows uses first and second PCR dispersions by way of example, although the present disclosure is not to be limited to systems having only two sample droplet generators producing first PCR dispersions and second PCR dispersions, as the system and method are suited for multiplexing of higher order systems having, for example, three, four, five, ten, twenty or more, e.g. a plurality of sample droplet generators, producing a plurality of PCR dispersions, wherein each PCR dispersion may be different to each and every one of the other PCR dispersions generated.

Once a sample and PCR Master Mix have been introduced into the microfluidic cartridge they may be pumped, using a pressure-driven fluid driver, from their respective inlets, or reservoirs to be mixed to form a first PCR mixture. The mixing may occur using a microfluidic mixer as described herein. However, it is also possible for a sample and PCR Master Mix to be mixed prior to be injected or inserted into the microfluidic cartridge. Once a first PCR mixture is present in the microfluidic cartridge, this is then partitioned, by a sample droplet generator in the microfluidic cartridge, into a plurality of aqueous droplets which are dispersed in a carrier liquid to form a first PCR dispersion. The carrier liquid may be any liquid capable of stably dispersing the aqueous droplets, for example as a water-in-oil emulsion. The carrier liquid may be any suitable oil, or other hydrophobic liquid. Examples of suitable oils include mineral oils used for PCR purposes, such as those used to prevent evaporation of aqueous systems during amplification.

The first PCR dispersion is then transported into a thermocycling chamber of the microfluidic cartridge in a flow of carrier liquid which is pumped to the thermocycling chamber. While these acts of dispersion and transportation have been described as being separate acts, in normal operation these will occur almost simultaneously, as the pump will initiate flow of carrier liquid toward thermocycling chamber, and the first PCR mixture will be dispersed into that carrier liquid and immediately transported toward the thermocycling chamber without the flow of carrier liquid being interrupted or paused.

As described above, in some examples a separator dye may be used, to physically separate the first PCR dispersion from the second PCR dispersion while in the thermocycling chamber, with the method including dispersing, in the microfluidic cartridge, a separator dye solution into the carrier liquid to form a dye dispersion, and transporting the dye dispersion into the thermocycling chamber after the first PCR dispersion.

Although each droplet of each of first and second PCR dispersions is a self-contained PCR vessel stable to thermocycling conditions, and each of first and second PCR dispersions can utilise a different reporter molecule, use of the separator dye in a droplet dispersion can facilitate processing and optical characterisation of the droplet dispersions after amplification. The separator dye may be another fluorescent reporter dye, but having a different fluorescence spectrum to the reporter dyes used in the first and second PCR dispersions. For example, the separator dye may be a rhodamine dye, while the first PCR dispersion includes a SYBR Green reporter and the second PCR dispersion includes a Cy5 reporter. Thus, in some examples of the method, once the first PCR dispersion has been transported into the thermocycling chamber, a separator dye dispersion may be generated, by dispersing a dye solution into the flow of carrier fluid to form a plurality of aqueous droplets containing the dye reporter molecule. The flow of carrier fluid can then transport the separator dye dispersion into the thermocycling chamber, behind the first PCR dispersion.

In other examples, a positive control nucleic acid is included within each PCR dispersion, for example via a PCR Master Mix. In addition to a reporter molecule to confirm a positive amplification of the target nucleic acid, each PCR dispersion may include a reporter probe specific to the positive control nucleic acid. For example, a PCR Master Mix for the first PCR dispersion may include a TaqMan probe having a first fluorescent dye, while a PCR Master Mix for the second PCR dispersion may include a a TaqMan probe having a second fluorescent dye which fluoresces at a different wavelength. Use of positive controls and associated TaqMan probes will result in fluorescence of the TaqMan dye once this has been cleaved from the probe, presenting an alternative to the separator dye dispersion to differentiate between the first and second PCR dispersions.

Once the first PCR dispersion (and, if being used, the separator dye dispersion) has been transported into the thermocycling chamber, a second PCR mixture is then processed. The second PCR mixture may be different to the first PCR mixture, in that it may contain a different nucleic acid sample, or it may contain a different PCR Master Mix with different primer pairs, for example. Regardless of how the second PCR mixture differs, the sample and PCR Master Mix for the second PCR mixture can be introduced into the microfluidic cartridge as described previously in connection with the first PCR mixture, i.e. separately, and mixed in the microfluidic cartridge, or they can be mixed prior to be introduced. More particularly, the second PCR mixture is partitioned, by a sample droplet generator in the microfluidic cartridge, into a second plurality of aqueous droplets dispersed into the carrier liquid to form a second PCR dispersion, which is then transported by pumping into the thermocycling chamber. If a separator dye dispersion is being used, then the second PCR dispersion will be separated from the first PCR dispersion by the separator dye dispersion.

During the method, once the thermocycling chamber has been completely filled with a plurality of different droplet dispersions or PCR dispersions to the exclusion of any air bubbles which can be expelled via a vent, no further fluid flow occurs in the thermocycling chamber. That is, subjecting the reaction mixture to conditions suitable for amplification by polymerase chain reaction comprises thermocycling in the absence of fluid flow within the thermocycling chamber

Once the first and second PCR dispersions, or a plurality of PCR dispersions have been introduced into the thermocycling chamber, heat is provided to raise the temperature of the reaction mixture of each droplet of the dispersions. In some examples, heat is provided by means of a heater in the form of a printed electrical trace provided in or on the substrate on which the thermocycling chamber is located.

Since the microfluidic cartridge is in thermal contact with the cooling module when present in the PCR system, when the temperature of the reaction mixture within the droplets needs to be adjusted, the cooling module, which is maintained at a temperature of no more than about 35° C., for example about 25° C., can extract heat from the droplets.

In some examples, the cooling module includes a working fluid or coolant, and the fluid is flowed through the cooling module to provide cooling and extract heat from the reaction mixture. In some examples, the fluid is selected from water, ethylene glycol and propylene glycol, and mixtures thereof, and is flowed through the cooling module at a flow rate of up to 4 L/min, for example from 1 L/min up to 4 L/min, for example from 1.1 L/min to 4 L/min, for example from 1.3 L/min to 3.5 L/min, for example from 1.5 L/min to 3.3 L/min, for example from 2 L/min to 3 L/min. While greater, or quicker, heat transfer may be achieved using a flow of working fluid through the cooling module, adequate heat transfer may also be achieved without any fluid flow. Thus, in some examples, the fluid is static in the cooling module.

The three basic steps of a single round of PCR amplification are denaturation, annealing and chain extension, each optimally taking place at different temperatures (for example in the ranges 94-98° C. for denaturation; 50-65° C. for annealing, and 70-80° C. for chain extension, depending on polymerase). A reaction emulsion of first and second PCR dispersions present in the thermocycling chamber of the microfluidic device described herein may therefore be heated by the heater in the microfluidic device to a denaturation temperature of from 94-98° C. for a sufficient time for any double stranded DNA in each reaction droplet to separate or denature into single stranded DNA. According to the thermocycling protocol described above, the reaction emulsion must then be cooled to an annealing temperature of from 50-65° C. While the turning off of a heater in thermal contact with the reaction emulsion will cease any further heating, it will not rapidly cool the reaction emulsion, as is desirable. However, with the introduction of the cooling module as described herein, heat can be dissipated away from the reaction emulsion to a fluid (for example water) flowing through the cooling module.

For each cycle of amplification, the duration of the denaturation step may account for 10-20% of the cycle duration, while the annealing step may account for 10-30% of the cycle duration and the extension step may account for 40-80% of the cycle duration. In some examples, the denaturation step may take from 0.1 to 3 seconds, and in some examples, 0.5 seconds. In some examples, the annealing step may take from 0.1 to 3 seconds, and in some examples, 0.5 seconds. In some examples the extension step may take from 1 to 60 seconds, and in some examples, from 5 to 10 seconds. In some examples, the nucleic acid is subjected to amplification conditions by PCR by thermocycling the reaction mixture for up to 50 cycles, for example from 10 to 50 cycles, or from 20 to 50 cycles, or about 40 cycles.

The conditions suitable for amplification by polymerase chain reaction may comprise providing heat from the heater to the thermocycling chamber to heat the reaction emulsion of first and second PCR dispersions at a heating rate of from 20° C./second to 200° C./second. The conditions suitable for amplification by polymerase chain reaction may comprise heating the reaction emulsion to a denaturing temperature of the nucleic acid of interest by providing a pulse of energy to a heater of the microfluidic cartridge. The conditions suitable for amplification by polymerase chain reaction may comprise pulse-controlled amplification, in which a time varying heat flux is applied to the reaction emulsion resulting in a time varying temperature gradient in the reaction mixture, as will be explained in more detail below.

While each of the temperatures associated with the three stages of PCR may be considered as a step of a thermocycle, each thermocycle necessarily also involves a heating step which begins before the denaturing step, but which may also overlap with the denaturing step. In this heating step, the temperature of the reaction mixture with respect to the starting temperature or the temperature of a previous elongation step is increased, in order to facilitate denaturing. Subjecting the reaction emulsion to conditions suitable for amplification by polymerase chain reaction may therefore comprise heating the reaction emulsion to a denaturing temperature of the nucleic acid of interest in each droplet by providing a pulse of energy to an embedded heater. Through the effect of the heater, a time varying heat flux is applied to the reaction mixture, resulting in a time varying temperature gradient in the reaction mixture, meaning a localised temperature of at least 90° C., for example at least 95° C., can rapidly be reached in the vicinity of the heater.

The length of time of heating is the total duration, in which pulses of energy are provided to the heater for it to transmit heat with a power suitable for denaturing of a nucleic acid sample, which may correspond to a transient, localised heating of the reaction mixture to a temperature of at least 90° C. The heating time is the total duration, in which pulses of energy are provided to the heater so that heat flows from the heater to the reaction emulsion to bring about a temperature increase that is suitable for denaturing.

The unravelling of a DNA double strand and diffusion of the two strands form one another (to avoid re-hybridization), can require a sufficiently high temperature to be maintained for a certain period of time. However, since the heater effects a localised heating, the duration of pulsed heating to effect denaturation may be less than 10 seconds, for example less than 5 seconds, for example less than 3 second, for example less than 1 second, for example less than 500 ms (milliseconds), for example less than 250 ms, for example less than 100 ms, for example less than 50 ms, for example less than 25 ms, for example less than 10 ms, for example less than 8 ms, for example less 3 ms, for example less than 1 ms, for example less than 500 μs (microseconds), for example less 300 μs, for example less than 100 μs, for example less than 50 μs, for example less than 30 μs, for example less than 10 μs.

For example, for a dispersion volume of 30 μL in a thermocycling chamber, increasing the temperature of an aqueous solution by up by 30° C. (as would be required to take a reaction droplet from a chain elongation temperature of 70-80° C. to a denaturation temperature of 90-95° C.) can be achieved by heating a single serpentine heater of etched copper with a pulse of energy having a heat flux of 4000 kW/m² for 1 ms (millisecond), with a pulse of energy having a heat flux of 400 kW/m² for 10 ms (milliseconds), or with a pulse of energy having a heat flux of 40 kW/m² for 100 ms (milliseconds). In another example, heating a single serpentine heater of etched copper having a footprint of 450 mm² by providing 115 W of DC power at a duty of 40% for less than, for example, 250 milliseconds as described above provides for a heating ramp rate to a denaturing temperature of 95° C. of 140° C./second, while a duty of 25% just as rapidly heats a liquid in the thermocycling chamber to an annealing temperature of 55° C. Thus, the heater is able to rapidly heat a small interfacial volume of reaction emulsion in the thermocycling chamber and rapidly and reliably effect denaturation.

As described earlier, the heater of the microfluidic cartridge may include a plurality of etched copper serpentine heaters, each provided in a different layer or plane of a substrate underlying the reaction chamber. For such a system, increasing the temperature of a reaction emulsion from a chain elongation temperature of 70-80° C. to a denaturation temperature of 90-95° C. can be achieved by heating the heaters with a pulse of energy having a combined heat flux of 4000 kW/m² for 1 ms (millisecond), with a pulse of energy having a combined heat flux of 400 kW/m² for 10 ms (milliseconds), or with a pulse of energy having a combined heat flux of 40 kW/m² for 100 ms (milliseconds).

As described earlier, the heater may include an embedded heater such as an etched copper trace overlaid with a diffuser layer or heat spreader of thermally conductive material. For such a system, increasing the temperature of a reaction emulsion from a chain elongation temperature of 70-80° C. to a denaturation temperature of 90-95° C. can be achieved by heating the heater with a heat flux of 4000 kW/m² for 1 ms (millisecond), with a heat flux of 400 kW/m² for 10 ms (milliseconds), or with a combined heat flux of 40 kW/m² for 100 ms (milliseconds) and allowing the diffuser layer to diffuse the generated heat into the thermocycling chamber.

Exact power requirements to bring a reaction mixture to an annealing temperature may vary for any given system. For example, required power input will depend on the dimensions of the heater, with required power being scalable with heater area, but may also depend on the presence or absence of a compliant thermal interface material, and whether or not the embedded heater is embedded in the substrate or fluidic stack so as to form an internal surface of the thermocycling chamber, or is fully embedded within a substrate layer.

Once denaturation has been effected, a cooling step begins before the annealing step, in order to reach the temperature required for annealing, usually 50-65° C. The cooling may take place through heat transfer to the cooling module, once power to the heater has been stopped. Since the cooling module is maintained at a temperature below the thermocycling temperatures, for example 35° C. or less, for example about 25° C., then rapid cooling rates of from 20° C./second to 100° C./second, for example about 30° C./second can be obtained.

Thus, the duration of cooling to effect annealing of the primers to the nucleic acid may be less than 10 seconds, for example less than 5 seconds, for example less than 3 second, for example less than 1 second, for example less than 500 ms (milliseconds), for example less than 250 ms, for example less than 100 ms, for example less than 50 ms, for example less than 25 ms, for example less than 10 ms, for example less than 8 ms, for example less 3 ms, for example less than 1 ms, for example less than 500 μs (microseconds), for example less than 300 μs, for example less than 100 μs, for example less than 50 μs, for example less than 30 μs, for example less than 10 μs.

Once annealing has been effected, a heating step begins before the chain extension or elongation step, in order to reach the temperature required for chain extension, usually 70-80° C.

The length of time of heating for chain extension is the total duration, in which the heater transmits heat with a power suitable for chain extension of a nucleic acid sample, which may correspond to a transient, localised heating of the reaction emulsion to a temperature of about 70-80° C. The heating time is the total duration, in which heat flows from the heater to the reaction emulsion to bring about a temperature increase suitable for chain extension in each droplet.

In one example, the annealing temperature is equal to the chain extension temperature. If the annealing temperature is equal to the chain extension temperature, only one temperature cycle with two different temperatures is required to amplify the nucleic acid of interest. The melt temperatures of the primers and the polymerase used may be selected so that at the primer melting temperature the polymerase used can still synthesize DNA at a sufficient speed. In this example, the temperature for annealing and chain extension is achieved by global heating and the denaturing step is achieved through localised heating.

The duration of heating to effect chain extension may be less than 10 seconds, for example less than 5 seconds, for example less than 3 second, for example less than 1 second, for example less than 500 ms (milliseconds), for example less than 250 ms, for example less than 100 ms, for example less than 50 ms, for example less than 25 ms, for example less than 10 ms, for example less than 8 ms, for example less 3 ms, for example less than 1 ms, for example less than 500 μs (microseconds), for example less 300 μs, for example less than 100 μs, for example less than 50 μs, for example less than 30 μs, for example less than 10 μs.

In one example, the heater comprises an array of 75 gold-coated tungsten wires (15 μm diameter, 200 nm Au coating) arranged on an internal surface of the thermocycling chamber. Localised heating of a layer of reaction emulsion of a few micrometers depth adjacent the wire is achieved via application of sub-millisecond voltage pulses to the wires. In particular, for providing localized heating for a chain extension step, a pulse at substantial peak power in the order of 1 kW for less than 500 microseconds is applied to the wire array from a 10 mF capacitator loaded to 30-40 V via a MOSFET (metal-oxide-semiconductor field-effect transistor; serving as a fast switch). This is sufficient to locally heat the solution to 60° C. to 80° C. for chain extension to occur.

In another example, localized heating for a chain extension step can be achieved by heating a thin film flat panel heater (tungsten film, 15 μm thickness with a 200 nm thick Au coating) with a heat flux of 4000 kW/m² for 1 ms (millisecond), with a heat flux of 400 kW/m² for 10 ms (milliseconds), or with a heat flux of 40 kW/m² for 100 ms (milliseconds) from a 10 mF capacitator loaded to 30-40 V via a MOSFET (metal-oxide-semiconductor field-effect transistor.

Thus, an embedded heater as described herein is able to rapidly heat a small volume of reaction emulsion in the thermocycling chamber and rapidly effect chain extension of the primers by the polymerase, using a nucleic acid of interest as the template, in each droplet of the PCR dispersions.

One example of a thermocycling protocol for a digital droplet PCR amplification of a plurality of droplets dispersed in a carrier oil in the thermocycling chamber may include:
operating the cooling module so as to maintain a substantially constant temperature of about 25° C.;
providing power to the heater to heat the droplet emulsion in the thermocycling chamber to a temperature of 90° C. to 100° C. at a ramp rate of 20° C./second to 200° C./second to allow denaturation to occur;
switching off power to the heater and actively cooling the droplet emulsion using the cooling module at a cooling rate of 20° C./second to 100° C./second to cool the reaction mixture to a temperature of 40° C. to 65° C. for annealing of the PCR primers to a target nucleic acid (if present in the sample); and
providing power to the heater to heat the droplet emulsion to a temperature of 60° C. to 80° C. at a ramp rate of 20° C./second to 100° C./second to allow chain extension to occur.
repeating the above steps for up to 40 cycles.

Following completion of the amplification protocol (for example 40 cycles of the above protocol), the pressure-driven fluid driver is reactuated, to once again cause flow through the microfluidic cartridge. Specifically, the pressure-driven fluid driver is actuated to successively transport each droplet of the first and second PCR dispersions from the thermocycling chamber to an optical readout zone of the microfluidic cartridge. As each droplet passes through the optical readout zone, the optical sensor (e.g. a fluorimeter) detects an optical signal from each droplet.

The first and second PCR dispersions may differ by virtue of the nature of the reporter molecule in each. Use of fluorescent reporter molecules which fluoresce at different wavelengths allows for identification and differentiation of the first and second PCR dispersions as each droplet of the dispersions is pumped through the optical readout zone following amplification. In other examples of the method, a separator dispersion of a separator dye dispersed into a plurality of aqueous droplets is formed as described, and so the optical sensor will detect different emission spectra for reporter molecules within the first and second PCR dispersions, and for the separator dye. A computer control module coupled to the optical sensor will then be able to determine the content of each droplet in turn, and thus be able to count the number of positive amplification results within each of the first and second PCR dispersions.

The digital droplet PCR system and method described are more tolerant to PCR inhibitors as they rely on endpoint detection rather than continuous detection, and so are not as sensitive to PCR kinetics which can be highly variable in the presence of PCR inhibitors. The digital droplet PCR system enables spatial multiplexing within a single chamber, yet has simple optical detection through sequential interrogation of each droplet in turn in the optical readout zone.

While the systems, methods and related aspects have been described with reference to certain examples, it will be appreciated that various modifications, changes, omissions, and substitutions can be made without departing from the spirit of the disclosure. It is intended, therefore, that systems, methods and related aspects be limited only by the scope of the following claims. Unless otherwise stated, the features of any dependent claim can be combined with the features of any of the other dependent claims, and any other independent claim.

The invention claimed is:

1. A digital droplet PCR system, comprising:
a microfluidic cartridge comprising:
a plurality of sample droplet generators, wherein each sample droplet generator is operable to partition a PCR mixture into a plurality of aqueous droplets dispersed in a carrier liquid, and wherein at least one sample droplet generator is operable to partition a PCR mixture that is different to a PCR mixture that is partitioned by at least one other sample droplet generator;
a thermocycling chamber comprising an embedded heater, an inlet configured to receive the plurality of aqueous droplets from the plurality of sample droplet generators, and an outlet; and
an optical readout zone fluidly connected to the outlet of the thermocycling chamber; and
a pressure-actuated pump configured to couple to and cause fluid flow through the microfluidic cartridge.

2. The digital droplet PCR system of claim 1, wherein the thermocycling chamber comprises an outlet which tapers toward the optical readout zone.

3. The digital droplet PCR system of claim 1, further comprising a reagent reservoir operatively coupled to at least one sample droplet generator, wherein a PCR Master Mix is provided in the reagent reservoir.

4. The digital droplet PCR system of claim 1, wherein each sample droplet generator is configured to disperse the plurality of aqueous droplets into the carrier fluid in a flow channel of the microfluidic cartridge, each droplet having a diameter at least 50% of the diameter of the flow channel.

5. The digital droplet PCR system of claim 1, wherein each sample droplet generator is fluidly connected to a respective sample reservoir and a respective reagent reservoir.

6. The digital droplet PCR system of claim 1, wherein each sample droplet generator is fluidly connected to a respective sample reservoir and a common reagent reservoir which is fluidly connected to at least one other sample droplet generator.

7. The digital droplet PCR system of claim 1, wherein each sample droplet generator is fluidly connected to a respective reagent reservoir and a common sample reservoir which is fluidly connected to at least one other sample droplet generator.

8. The digital droplet PCR system of claim 1, further comprising a separator dye reservoir fluidly connected to a separator droplet generator.

9. A method of performing PCR, comprising:
partitioning, in a microfluidic cartridge, a first PCR mixture into a plurality of aqueous droplets and dispersing the plurality of aqueous droplets in a carrier liquid to form a first PCR dispersion;
transporting the first PCR dispersion into a thermocycling chamber of the microfluidic cartridge;
partitioning, in the microfluidic cartridge, a second PCR mixture into a plurality of aqueous droplets and dispersing the plurality of aqueous droplets in a carrier liquid to form a second PCR dispersion;
transporting the second PCR dispersion into the thermocycling chamber to form a reaction emulsion of first and second PCR dispersions;
subjecting the reaction emulsion to conditions suitable for amplification using polymerase chain reaction; and
successively transporting each droplet of the reaction emulsion from the thermocycling chamber to an optical readout zone of the microfluidic cartridge and detecting an optical signal from each droplet.

10. The method of claim 9, wherein the first PCR mixture and the second PCR mixture each comprise a sample suspected of containing a nucleic acid of interest, and a PCR Master Mix.

11. The method of claim 9, wherein the first PCR mixture differs from the second PCR mixture by having a different nucleic acid sample for amplification, or a different PCR Master Mix.

12. The method of claim 9, further comprising:
dispersing, in the microfluidic cartridge, a separator dye solution into the carrier liquid to form a dye dispersion, and transporting the dye dispersion into the thermocycling chamber after the first PCR dispersion.

13. The method of claim 9, wherein the carrier liquid comprises an oil.

14. The method of claim 9, further comprising:
including in the first PCR dispersion and/or the second PCR dispersion a positive control nucleic acid and a reporter probe specific the positive control nucleic acid.

15. The method of claim 9, wherein the microfluidic cartridge is coupled to a pressure-actuated pump configured to pump carrier liquid through a flow channel to the thermocycling chamber, and to respectively pump the first PCR mixture and the second PCR mixture to the flow channel so as to form the first and second PCR dispersions.
